(19) United States
(12) Patent Application Publication
(10) Pub. No.: US 2022/0167893 A1
(43) Pub. Date: Jun. 2, 2022

(54) METHODS AND SYSTEMS FOR DETECTING INDICATIONS OF COGNITIVE DECLINE

(71) Applicant: KONINKLIJKE PHILIPS N.V., EINDHOVEN (NL)

(72) Inventors: Gerhard Reinhold SPEKOWIUS, Straelen (DE); Koray KARAKAYA, Eindhoven (NL)

(21) Appl. No.: 17/539,675

(22) Filed: Dec. 1, 2021

Related U.S. Application Data
(60) Provisional application No. 63/120,264, filed on Dec. 2, 2020.

(51) Int. Cl. A61B 5/16; G16H 20/70; A61B 5/00

(52) U.S. Cl. CPC A61B 5/16; G16H 20/70; A61B 5/6804; A61B 5/742; A61B 5/0022; A61B 5/7475

(57) ABSTRACT

Systems and methods for detecting and managing cognitive decline of a person using a cognitive training device are provided involving a first information source having a device including a sensor configured to detect sensor data corresponding to a first behavior of the person, wherein the first behavior represents a first deviation from a first expected pattern; a second information source configured to receive, store, and transmit data corresponding to a second behavior of the person, wherein the data indicates the second behavior represents a second deviation from a second expected pattern; and a processor configured to analyze the sensor data and the data to determine whether a predetermined threshold has been reached indicating the person is experiencing a first stage of cognitive decline, and generate actionable information to assist a user in determining a treatment for the person to delay the progression of the first stage of cognitive decline.

### CROSS-REFERENCE TO PRIOR APPLICATIONS

This application claims the benefit of U.S. Provisional Application No. 63/120264, filed on Dec. 2, 2020. This application is hereby incorporated by reference herein.

### FIELD OF THE DISCLOSURE

The present disclosure is directed generally to methods and systems for detecting indications of cognitive decline in an automated manner and delaying the progression of cognitive decline. More specifically, the present disclosure is directed generally to providing automated detection of memory decline across different platforms to assist non-clinical caregivers, informal clinical caregivers, and healthcare professionals care for people with memory issues.

### BACKGROUND

Although people experience normal cognitive decline as they age, more serious cognitive decline can indicate dementia or some other condition. People who experience more memory issues than normal for people their age can be diagnosed with mild cognitive impairment which can be a stage between the expected cognitive decline associated with aging and the more serious decline of dementia. Early detection of mild cognitive impairment can facilitate early intervention and potentially delay dementia. However, detecting symptoms of mild cognitive impairment can be challenging. Subjective cognitive decline is the self-reported experience of worsening memory issues. A person may notice that their own memory has diminished. Family and friends may also notice changes in a person's memory. Typically, when seeking an evaluation of cognitive impairment, the person suspicious of their own cognitive decline or a family member or friend must recall the person's forgetfulness by answering general questions, such as, “Do you/Does he/she remember future and past events?”. The answers are typically limited to dichotomous scales (e.g., yes/no) or rating scales (e.g., always/often/sometimes/seldom/never). Yet, this information does not convey when and what specifically the care recipient had forgotten. The information also typically does not convey if the instances of forgetfulness are increasing or decreasing in frequency. Additionally, the answers that are provided via evaluative questionnaires are provided after the fact and thus, are more prone to error.

Thus, there is a need in the art for improved methods and systems for automatically detecting instances of memory decline for a person with subjective cognitive decline.

### SUMMARY OF THE DISCLOSURE

The present disclosure is directed to inventive methods and systems for automated detection and management of cognitive decline of a person. Generally, embodiments of the present disclosure are directed to improved methods and systems for automatically detecting cognitive decline using a plurality of devices. Applicant has recognized and appreciated that it would be beneficial to extract information pertaining to instances of forgetfulness that are recorded in real time using a plurality of devices. Various embodiments and implementations herein are directed to retrieving information from at least two different information sources regarding instances when a person potentially experienced an instance of forgetfulness that may be indicative of memory decline, analyzing the information to determine whether a predetermined threshold has been reached indicating the person is experiencing a first stage of cognitive decline corresponding with subject cognitive decline or mild cognitive impairment; and generating actionable information to assist a user (e.g., an informal caregiver or a healthcare professional) in determining an intervention (e.g., treatment) for the person to delay progression of the first stage of cognitive decline.

Generally, in one aspect, a system for managing cognitive decline of a person is provided. The system includes a first information source having a device having a sensor configured to detect sensor data corresponding to a first behavior of the person and a first processor configured to determine, based on the sensor data, that the first behavior represents a first deviation from a first expected pattern, wherein the device is further configured to store and transmit the sensor data; a second information source configured to receive, store, and transmit data corresponding to a second behavior of the person, wherein the data indicates the second behavior represents a second deviation from a second expected pattern; an input configured to receive the sensor data from the first information source and the data from the second information source; a database configured to store the sensor data and the data from the first and second information sources; and a second processor in communication with the first and second information sources and the database, wherein the second processor is configured to: (i) analyze the sensor data and the data to determine whether a predetermined threshold has been reached indicating the person is experiencing a first stage of cognitive decline, and (ii) generate actionable information to assist a user in determining a treatment for the person to delay the progression of the first stage of cognitive decline.

In embodiments, the first expected pattern includes a completion of a task and the first deviation includes the person's failure to complete the task in its entirety.

In embodiments, the second information source includes a user input device and a processor configured to compare input from the person with input from the user and determine whether the inputs from the user and the person match.

In embodiments, the processor is configured to request the sensor data be transmitted from the first information source to the database based on a frequency at which the person uses the device.

In embodiments, the system further includes a graphical display in communication with the processor, wherein the graphical display is configured to present the generated actionable information. In embodiments, the graphical display is further configured to present a plurality of questions to the person and the processor is configured to analyze answers to the plurality of questions provided by the person to determine a memory strategy module for the person. In embodiments, the memory strategy module is configured to stimulate neural activity in the person.

In embodiments, the first expected pattern includes an anticipated pattern of use of the device and the first deviation includes the person's use of the device in an unanticipated manner.

In embodiments, the first expected pattern includes a first rate of use of the device that is consistent with a predetermined rate and the first deviation includes the person's use of the device at a second rate that is inconsistent with the first rate.

In embodiments, the device is part of an internet of things system where the device is integrated within an appliance, furniture, and/or clothing of the person.

Generally, in another aspect, a method for managing cognitive decline of a person using a cognitive training device, the method comprising the steps of: communicating, by a first processor, with a first information source including a device having a sensor configured to detect sensor data corresponding to a first behavior of the person, wherein the first behavior represents a first deviation from a first expected pattern; communicating, by the first processor, with a second information source configured to receive, store, and transmit data corresponding to a second behavior of the person, wherein the data indicates the second behavior represents a second deviation from a second expected pattern; receiving, by the first processor, the sensor data from the first information source and the data from the second information source and storing the sensor data and data in a database; analyzing, by the first processor, the sensor data and the data to determine whether a predetermined threshold has been reached indicating the person is experiencing a first stage of cognitive decline; generating, by the first processor, actionable information to assist a user in determining a treatment for the person to delay the progression of the first stage of cognitive decline; determining, by the first processor, a memory strategy module as part of the treatment for the person based on answers to a plurality of questions provided by the person; and implementing, by a memory strategy device, the memory strategy module to the person.

In embodiments, the memory strategy module is configured to stimulate neural activity in the person.

In embodiments, the first processor is further configured to request the sensor data be transmitted from the first information source to the database based on a frequency at which the person uses the device.

In embodiments, the second information source includes a user input device and a second processor configured to compare input from the person with input from the user and determine whether the inputs from the user and the person match.

In embodiments, the first expected pattern includes a completion of a task, an anticipated pattern of use of the device, or a first rate of use of the device that is consistent with a predetermined rate, and the first deviation includes the person's failure to complete the task in its entirety, the person's use of the device in an unanticipated manner, or the person's use of the device at a second rate that is inconsistent with the first rate.

In various implementations, the one or more processors described herein may take any suitable form, such as, one or more processors or microcontrollers, circuitry, one or more controllers, a field programmable gate array (FGPA), or an application-specific integrated circuit (ASIC) configured to execute software instructions. Memory associated with the processor may take any suitable form or forms, including a volatile memory, such as random-access memory (RAM), static random-access memory (SRAM), or dynamic random-access memory (DRAM), or non-volatile memory such as read only memory (ROM), flash memory, a hard disk drive (HDD), a solid-state drive (SSD), or other non-transitory machine-readable storage media. The term “non-transitory” means excluding transitory signals but does not further limit the forms of possible storage. In some implementations, the storage media may be encoded with one or more programs that, when executed on one or more processors and/or controllers, perform at least some of the functions discussed herein. It will be apparent that, in embodiments where the processor implements one or more of the functions described herein in hardware, the software described as corresponding to such functionality in other embodiments may be omitted. Various storage media may be fixed within a processor or may be transportable, such that the one or more programs stored thereon can be loaded into the processor so as to implement various aspects as discussed herein. Data and software, such as the algorithms or software necessary to analyze the data collected by the tags and sensors, an operating system, firmware, or other application, may be installed in the memory.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. These and other aspects of the various embodiments will be apparent from and elucidated with reference to the embodiment(s) described hereinafter.

### BRIEF DESCRIPTION OF THE DRAWINGS

In the drawings, like reference characters generally refer to the same parts throughout the different views. Also, the drawings are not necessarily to scale, emphasis instead generally being placed upon illustrating the principles of the various embodiments.

FIG. 1 is a schematic illustration of a system for detecting and managing cognitive decline of a person in accordance with aspects of the present disclosure;

FIG. 2 is an example process of detecting and managing cognitive decline of a person in accordance with aspects of the present disclosure; and

FIG. 3 is an example flow chart showing steps of an exemplary implementation of the method of FIG. 2 in accordance with aspects of the present disclosure.

### DETAILED DESCRIPTION OF EMBODIMENTS

The present disclosure describes various embodiments of methods and systems for improved automated detection of cognitive decline in a person using a plurality of devices. More specifically, Applicant has recognized and appreciated that it would be beneficial to use a variety of separate sources to collect data of a person's behaviors in real time to determine whether the person is experiencing cognitive decline or worsening cognitive decline. Additionally, Applicant has recognized and appreciated that it would be beneficial to collect more detailed data surrounding a person's behaviors to assist a user (e.g., an informal caregiver or a healthcare professional) in determining whether the person is experiencing cognitive decline or worsening cognitive decline and how to care for the person. Exemplary goals of utilization of certain embodiments of the present disclosure are to extract data from sources used by an informal or formal caregiver in caring for a person with memory issues and data from sources used by the person with memory issues to glean additional information as to the type of memory issues the person is experiencing and as to whether the memory issues are worsening.

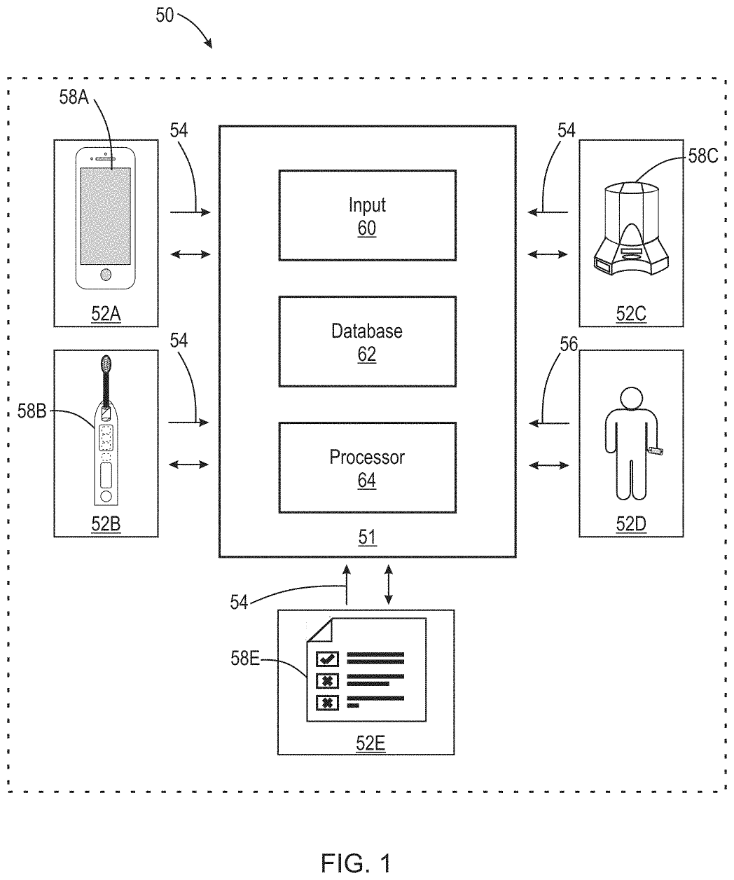

Referring to FIG. 1, a schematic depiction of a system 50 for detecting and managing cognitive decline of a person using a cognitive training device 51 is provided. The cognitive training device 51 can be any suitable device, such as, a mobile handheld device, e.g., a mobile phone, a personal computer, a laptop, a tablet, or any suitable device capable of receiving user input and executing and displaying a computer program product in the form of a software application or a platform. The application or platform can include any suitable cognitive evaluative procedure and/or compensatory cognitive training procedure, e.g., a cognitive training application. The cognitive detection system 50 further includes at least one information source 52A, 52B, 52C, 52D, and 52E configured to detect in real time whether the person has exhibited a behavior pattern that represents a deviation from an expected pattern. The information sources can be configured to record behavior patterns of the person as described below. In embodiments, the information sources are configured to store the recorded behavior patterns locally in a memory associated with the information source. In alternate embodiments, the information sources are configured to store the recorded behavior patterns at a remote device configured with a memory for storage. The cognitive detection system 50 further includes an input 60, a database 62, and a processor and an associated memory 64 within or coupled to the cognitive device 51. The processor 64 is configured to receive one or more types of data 54, 56 from the information sources via the input 60 and database 62. In embodiments, the processor 64 is configured to receive sensor data 54 from one or more sensor devices 58A, 58B, 58C, and 58E and data 56 via the input 60 and the database 62. The data 54, 56 indicate different behavior patterns of the person that deviate from expected or normal behavior patterns of the person. System 50 is intended to be utilized by a user, e.g., a caregiver or a healthcare professional who is providing care to the person experiencing cognitive decline.

It should be appreciated that the data described herein include any information that can be used to characterize the person's behavior as abnormal. For example, the data can include information describing how the person uses particular devices, such as, a toothbrush, a medication dispenser, a mobile phone, a smart coffee maker, mobile help devices such as devices used to help when a person is lost, and/or home automation devices. The data can also include information describing the person's activities including, but not limited to, exercising, sleeping time, time talking with family and friends, time attending social events, and time attending medical appointments. The data can also include information describing how the user (e.g., an informal caregiver or a healthcare professional) perceives the person and/or information gathered from the person via some assessment mechanism. The data can also include or be combined with physiological data of the person. The devices of the system 50 can be connected via any suitable Internet of Things (IoT) system.

As shown in FIG. 1, the information sources 52A, 52B, 52C, 52D, and 52E can be devices used by the person and/or the caregiver. In embodiments, information source 52A can be embodied as any suitable device 58A capable of receiving user input and executing and displaying a computer program product in the form of a software application or a platform. The software application includes a user interface configured to receive and/or display information useful to the person and/or the user as described herein. In an example, the software application is an online collaboration application that enables people to easily form and activate a care circle of trusted family and friends, access meaningful insights into their loved one's wellbeing, and receive notifications about their care, such as, the Philips Cares digital application. However, it should be appreciated that the disclosure is not limited to these embodiments, and thus the disclosure and embodiments disclosed herein can encompass any suitable platform. The device 58A of information source 52A includes a controller with a processor and a memory which can store an operating system as well as sensor data. The device 58A can also include a power source which can be AC power, or can be battery power from a rechargeable battery. In an embodiment, the person experiencing cognitive decline and/or the user providing care to the person uses device 58A to track certain tasks and/or activities that are to be completed and/or performed. For example, a task to be completed can include uploading a picture by a certain date. The device 58A can be coupled to the person's electronic calendar to track this information in embodiments. In other embodiments, the person and/or the caregiver can provide this information manually to the device 58A. In still other embodiments, the device 58A can retrieve such data from the person's electronic calendar. Device 58A can be configured to detect automatically whether each of the recorded or scheduled tasks or activities has been completed or performed or not. For example, the device 58A can sense if any documents such as a picture have been uploaded and stored on the local or remote memory. Alternatively, the device 58A can prompt the user to upload the picture until the task is completed and device 58A can sense if the prompt is responded to or not. Device 58A can include a clock and can record timing information associated with when the user uploads the picture and/or responds to the prompt. In this way, the platform of device 58A can be used to automatically determine when the person has neglected to complete a scheduled task or activity. Since the device already knows what the scheduled task or activity was, it is particularly advantageous for the device 58A to provide data regarding what was missed and when it was missed to the processor 64 of system 50.

The device 58A can also include a connectivity module configured and/or programmed to transmit data to a wireless transceiver of the device 51. In embodiments, the connectivity module of the device 58A can transmit data via a Wi-Fi connection over the Internet or an Intranet to database 62 or some other location. Alternatively, the connectivity module may transmit sensor data via a Bluetooth or other wireless connection to a local device (e.g., a separate computing device), database 62, or other transceiver. For example, the connectivity module can transmit data to a separate database to be stored, to transmit data for further analysis, or to share data with other users.

The software application of device 58A can be a web-based or network-based application, e.g., an application stored and executed on a remote server over the internet; however, it should be appreciated that the software application can also be a native application, e.g., an application that is stored and executed on local memory of device 58A and does not require a connection to the internet to function. Thus, it should be appreciated that the instructions and functionality related to the execution and use of the software application may be stored and executed on device 58A, i.e., using a processor and an associated memory of the device 58A, or can be stored and executed using a remote server over the internet. Although device 58A is depicted as a mobile phone, it should be appreciated that information source 52A can also be embodied as a mobile personal computer comprising a CPU, a display monitor, keyboard, and mouse as separate components, a laptop comprising a CPU, a display monitor, keyboard, and a mouse as integrated components, a tablet, a desktop personal computer, or a computer program product in the form of a software application or a platform accessible over the internet.

In another embodiment, the person experiencing cognitive decline uses device 58A in communication with a wearable device configured to be worn by the person. The wearable device can be configured to locate and track the person and record the person's whereabouts either on the wearable device or on device 58A. The person or a user providing care to the person can record scheduled appointments in device 58A or device 58A can gather this information from one or more linked electronic calendars, and device 58A can use the location information from the wearable device to determine whether the person attended a scheduled appointment or missed the appointment since the person was not present where the scheduled appointment was scheduled to take place at the appropriate time. The device 58A can record all instances of missed appointments including what was missed, when they were missed, and where they were missed. In embodiments, the device 58A can record where the person was located when they missed an appointment as well. The wearable device can use suitable global positioning systems (GPS), assisted or augmented GPS systems, Wi-Fi locating systems, or intelligent locating systems. In an example embodiment, the wearable device uses intelligent locating systems to record regularly visited locations and/or locations expected to be visited based on data received from device 58A, and the wearable device can determine whether the person is expected to miss an upcoming appointment. The wearable device can also be configured to track physiological data of the person, for example, via one or more sensors configured to detect the person's heart rate, blood pressure, or temperature. It should be appreciated that any physiological data can be measured or derived using a suitable wearable device.

Information source 52B can be embodied as any suitable personal care device 58B that is configured to record the person's usage of the device 58B and compare the person's usage to an intended or programmed use. The embodiments and implementations disclosed or otherwise envisioned herein can be utilized with any personal care device. Examples of suitable personal care devices include a toothbrush, such as a Philips Sonicare® toothbrush (manufactured by Koninklijke Philips, N.V.), a flossing device such as a Philips AirFloss® device, an oral irrigator, a tongue cleaner, or other personal care device. However, the disclosure is not limited to these enumerated devices, and thus the disclosure and embodiments disclosed herein can encompass any personal care device. An example suitable personal care device 58B includes a controller with a processor and a memory which can store an operating system as well as sensor data. The device 58B also includes a power source which can be AC power, or can be battery power from a rechargeable battery. The sensor of the device 58B can be an inertial motion sensor such as an accelerometer, gyroscope, or magnetic sensor configured to generate sensor data in response to motion and communicate that data to the controller of the device. The controller of the device can analyze the data and determine whether the person's usage of the device constitutes a deviation from the person's expected usage of the device. The person's expected usage of the device can be based on historical sensor data in embodiments and/or trends in the historical sensor data. The deviation must be significant enough to account for a meaningful difference in usage. Thus, the controller of the device can determine whether the deviation meets or exceeds a predetermined threshold value.

The device 58B can also include a connectivity module configured and/or programmed to transmit sensor data to a wireless transceiver of the device 51. In embodiments, the connectivity module of the device 58B can transmit sensor data via a Wi-Fi connection over the Internet or an Intranet to database 62 or some other location. Alternatively, the connectivity module may transmit sensor data via a Bluetooth or other wireless connection to a local device (e.g., a separate computing device), database 62, or other transceiver. For example, the connectivity module can transmit sensor data to a separate database to be stored, to transmit sensor data for further analysis, to transmit user feedback to a separate user interface, or to share data with other users.

Information source 52C can be embodied as any suitable medication dispenser 58C configured to record the person's usage of the device 58C and compare the person's usage to an intended or programmed use. An example of a suitable medication dispenser includes an automated medication dispensing device 58C that can be loaded with single-dose pill cups. The medication dispensing device 58C can also include a controller with a processor and a memory which can store an operating system as well as sensor data. The device 58C also includes a power source which can be AC power, or battery power from a rechargeable battery. The device 58C further includes a user interface which is configured to transmit or receive information to the user. The device 58C can also include a clock and/or timer. The memory can receive and store a medication dispensing schedule and the processor can generate a visual and/or audible alarm to indicate when it is time for the person to take a dose of medication. In embodiments, the device 58C can additionally or alternatively transmit a notification to be displayed on a user interface of a mobile device associated with the person taking the medication. To take the medication as prescribed at the appropriate time, the person can press a release button on the device 58C or the mobile device to dispense a single dose. A sensor of the device 58C or mobile device can determine if the release button has not been pressed within a predetermined amount of time from the time when the dose should have been taken and communicate that data to the controller of the device 58C. In embodiments, each time that the person misses a dose of medication, the device 52C can transmit data related to the missed dose directly to the processor 64 of system 50. In embodiments, the device 52C can store the data related to the missed doses locally and the processor 64 of the system 50 can request such data on a suitable regular basis.

Information source 52D can be embodied as the user, e.g., the caregiver providing care to the person experiencing cognitive decline, where the user can witness the person experiencing memory decline firsthand and record such observations directly to the database 62 of the system 50 in real time or within a short time period after. In embodiments, the caregiver can also provide observations of the person experiencing memory decline that were witnessed by someone else. The data provided by the caregiver includes information describing what the instance of forgetfulness was and when it happened. It should be appreciated that the caregiver can be the spouse of the person experiencing cognitive decline, another family member of the person experiencing cognitive decline, or a close friend of the person experiencing cognitive decline. The caregiver need not have any medical training.

Information source 52E can be embodied as a personalized test that indicates memory decline experienced by the person. For example, information source 52E can include a device 58E that is capable of receiving user input and executing and displaying a personalized test in the form of a software application or a platform. Device 58E can include a controller with a processor and a memory which can store an operating system as well as input and output data. The application of the device 58E can include a user interface configured to display the personalized test to the person. The personalized test can include questions regarding details from the person's recent past, e.g., what the person had for dinner the previous night. In embodiments, the user providing care to the person provides the details to the information source 52E, e.g., that the person had fish tacos for dinner the previous night. The user interface of the software application can be configured to receive the person's responses to the questions and the processor of the device 58E can analyze the person's answers and compare them with the details provided by the caregiver. If the person's answers match the details provided by the caregiver, the processor can determine that the person was able to accurately recall the details. If the answers do not match the details provided by the caregiver, the processor can determine that the person was unable to accurately recall the details. The controller of the device 58E can also include a connectivity module configured and/or programmed to transmit data to a wireless transceiver of the processor 64 of the system 50. The data transmitted by the connectivity module can include each question and answer of the personalized test that did not match.

It should be appreciated that although there are only five information sources shown in FIG. 1, any number of information sources is contemplated. Additionally, it should be appreciated that the information sources described are illustrative only and are not intended to limit the scope of the disclosure.

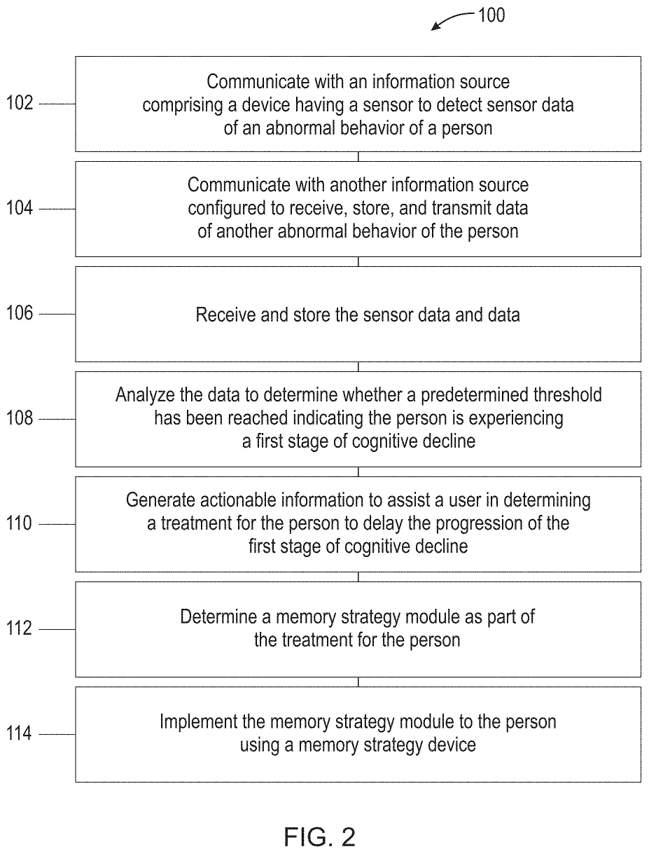

In FIG. 2, an example process 100 of detecting and managing cognitive decline of a person using a cognitive training device is provided. An example implementation of compensatory cognitive training using the foregoing systems begins with providing a cognitive training device having a processor and coupling the cognitive training device with a plurality of information sources. The plurality of information sources can be any of the sources described herein or any others. Each of the information sources automatically detect when the person experiences memory decline. For example, device 58A of information source 52A can be configured to automatically determine when the person has failed to complete a task or activity in its entirety. Device 58B of information source 52B can be configured to automatically determine when the person uses the personal care device 58B in an unanticipated manner where the unanticipated manner is sufficiently different. Device 58C of information source 52C can be configured to automatically determine when the person misses one or more doses of medication or attempts to take multiple doses prematurely, or otherwise uses the medication dispenser at a rate that is inconsistent with a prescribed rate. Information source 52D can record instances of forgetfulness, e.g., when the person forgets to contact the caregiver as planned. Device 58E of information source 52E can be configured to automatically detect when the person cannot recall details of recent events using a personalized test. Once an instance of forgetfulness is detected by an information source, the instance can be recorded locally.

At step 102, the processor of the cognitive training device communicates with at least one information source of a plurality of information sources to request data corresponding to a behavior of the person where the behavior represents a meaningful deviation. In embodiments, the processor of the cognitive training device can be configured to request data from the information source on a regular basis, e.g., daily, weekly, monthly, quarterly, depending on the type of information source and the type of deviated behavior being detected by the information source. For example, if the device 58A of information source 52A is configured to track missed medical appointments, the processor can be configured to request data on a regular monthly basis. In contrast, since the personal care device 52B and/or the medication tracking device 52C track patterns of use and/or rate of use of the devices, respectively, the processor can be configured to request data more frequently, such as, weekly or daily. It should be appreciated that in embodiments, the processor can be configured to request data from any of the information sources on an irregular basis as well. For example, the processor can be configured to request data from the information sources based on when the person has scheduled medical appointments or based on abnormal behaviors detected from other information sources. Thus, if the personal care device and/or the medication tracking device detect abnormal usage patterns, the processor of the cognitive training device can be triggered to request data from other information sources at a higher frequency.

At step 104, the processor of the cognitive training device communicates with another information source of a plurality of information sources to request data corresponding to another behavior of the person where the behavior represents a deviation. The discussion pertaining to step 102 also applies to step 104.

At step 106, the processor of the cognitive training device receives the sensor data from the information source of step 102 and data from the information source of step 104. The data from the information sources of steps 102 and 104 is received via the input 60 and stored in database 62 of system 50. It should be appreciated that database 62 can be integral with the processor, connected to the processor within the same device, or separate from the processor.

At step 108, the processor of the cognitive training device analyzes the data from the information sources of steps 102 and 104 to determine whether a predetermined threshold has been reached indicating the person is experiencing a first stage of cognitive decline corresponding with subjective cognitive decline. Any suitable algorithm can be used to analyze the data depending on the type of data and thresholds. In embodiments, the data can be analyzed to determine if the person is experiencing a steady increase in frequency of instances of forgetfulness generally and above a predetermined threshold frequency value. In embodiments, the analysis of the data can involve applying different weights to the data depending on the significance of particular types of data. For example, a single instance of the person forgetting to brush her teeth may not be weighted but, a single instance of the person forgetting his/her own anniversary might be weighted more heavily.

At step 110, the processor of the cognitive training device generates actionable information based on the results of the analysis at step 108. The generated actionable information is designed to assist a user in determining a treatment for the person to delay the progression of the first stage of cognitive decline. In embodiments, the generated actionable information is designed to alarm informal caregivers and/or healthcare professionals if additional measures are needed. For example, the actionable information can be any suitable indicator that the person has experienced a statistically significant increased level of forgetfulness. The indicator can be color-coded to indicate whether the increased level of forgetfulness is mild, intermediate, or substantial or serious in an embodiment. In embodiments, green can be used to indicate a mildly increased level of forgetfulness, yellow can be used to indicate a moderately increased level of forgetfulness, and red can be used to indicate a substantially increased level of forgetfulness. It should be appreciated that different colors may be used and/or different visual indicia. It should also be appreciated that different levels of forgetfulness can also be generated in the actionable information. Based on the actionable information, the user can provide the person with an appropriate questionnaire for triage. The answers provided by the person from the triage questionnaire can be input into the cognitive training device for further analysis.

At step 112, the processor of the cognitive training device determines a memory strategy module as part of the treatment for the person based on the answers provided by the person from the triage questionnaire. The memory strategy module can be configured to stimulate neural activity in the person in a particular manner depending on the particular stage of cognitive decline the person is determined to be experiencing.

At step 114, the memory strategy module assigned by the processor is implemented by a memory strategy device. The memory strategy device can be part of the cognitive training device or separate. In embodiments, the person can set goals for him/herself as part of the memory strategy module using the memory strategy device.

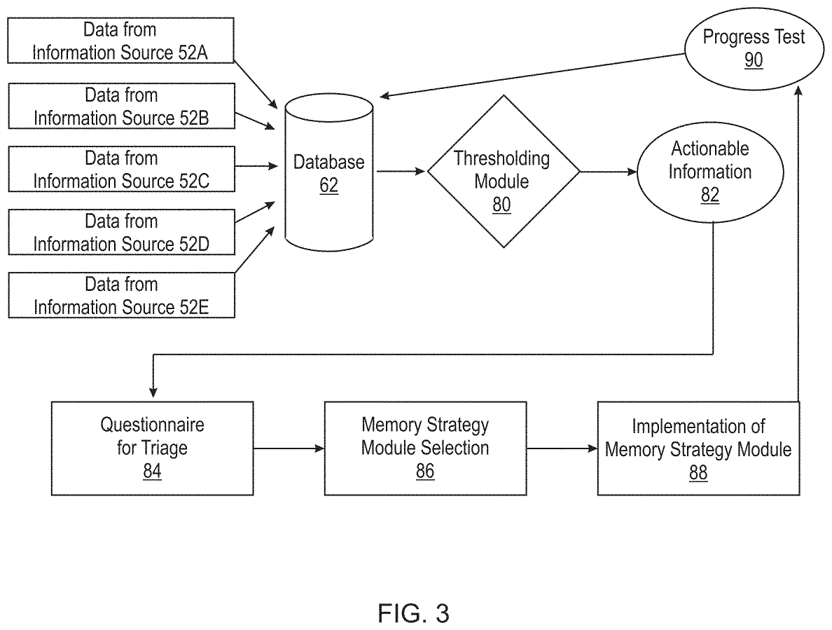

Referring to FIG. 3, an example flow chart showing steps of an exemplary implementation of a method of detecting and managing cognitive decline in a person is provided. The flow chart begins with data from a plurality of sources, e.g., 52A, 52B, 52C, 52D, and 52E being transmitted, received, and stored in a database e.g., 62. The data can be any information indicative of abnormal behavior of the person. The data can be retrieved from the database and run through a thresholding module 80 to determine if a predetermined threshold has been reached. The predetermined threshold can be any suitable value that defines a minimum number and type of memory issues that constitute mild cognitive decline rather than normal cognitive decline. In embodiments, the predetermined threshold can also account for when the memory issues occurred and can represent any suitable value that defines a minimum number and type of memory issues transpiring over a predefined period of time that constitute mild cognitive decline rather than normal cognitive decline.

If the analyzed data indicates the person is experiencing a first stage of cognitive decline, actionable information 82 can be generated to assist a user in determining a treatment for the person to delay the progression of the first stage of cognitive decline. Examples of actionable information can include any visible indicia corresponding to a level of forgetfulness that exceeds a certain threshold value or an increased level of forgetfulness.

Based on the level of forgetfulness indicated, the user providing care to the person can provide the person with any suitable questionnaire for triage 84. The answers to the questionnaire provided by the person can be used by the caregiver to assign a memory strategy module 86 as part of the treatment for the person. In embodiments, the answers to the questionnaire can be provided to the cognitive training device 51 and the device can assign a memory strategy module 86 for the person. The memory strategy module can be implemented by a memory strategy device to the person 88. In embodiments, the memory strategy device is integrated within the cognitive training device 51. In alternate embodiments, the memory strategy device is separate from yet in communication with the cognitive training device 51. The memory strategy module is configured to stimulate neural activity in the person depending on the person's level of cognitive decline.

In embodiments, the person can subsequently take progress tests 90 to see if the person's cognitive decline is worsening. The progress tests 90 can be provided by the person in embodiments. In alternate embodiments, the progress tests 90 can be provided by the memory strategy device and/or the cognitive training device 51. In embodiments, the results of the progress tests that indicate instances of forgetfulness can be stored in the database 62 of the system 50 and used as inputs for systems and methods described herein.

Using the methods and systems described herein, informal caregivers and healthcare professionals can access detailed and timely information of memory decline of a person when determining what additional measures are required to take care of the person and to keep them safe. The caregivers and professionals are better equipped to manage a person's cognitive decline when having knowledge of when and what the person forgets and whether the forgetfulness is increasing.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively.

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

What is claimed is:

1. A system for managing cognitive decline of a person, comprising:
a first information source comprising a device having a sensor configured to detect sensor data corresponding to a first behavior of the person and a first processor configured to determine, based on the sensor data, that the first behavior represents a first deviation from a first expected pattern, wherein the device is further configured to store and transmit the sensor data;
a second information source configured to receive, store, and transmit data corresponding to a second behavior of the person, wherein the data indicates the second behavior represents a second deviation from a second expected pattern;
an input configured to receive the sensor data from the first information source and the data from the second information source;
a database configured to store the sensor data and the data from the first and second information sources; and
a second processor in communication with the first and second information sources and the database, wherein the second processor is configured to: (i) analyze the sensor data and the data to determine whether a predetermined threshold has been reached indicating the person is experiencing a first stage of cognitive decline, and (ii) generate actionable information to assist a user in determining a treatment for the person to delay the progression of the first stage of cognitive decline.

2. The system of claim 1, wherein the first expected pattern comprises a completion of a task and the first deviation comprises the person's failure to complete the task in its entirety.

3. The system of claim 1, wherein the second information source comprises a user input device and a processor configured to compare input from the person with input from the user and determine whether the inputs from the user and the person match.

4. The system of claim 1, wherein the processor is configured to request the sensor data be transmitted from the first information source to the database based on a frequency at which the person uses the device.

5. The system of claim 1, further comprising a graphical display in communication with the processor, wherein the graphical display is configured to present the generated actionable information.

6. The system of claim 6, wherein the graphical display is further configured to present a plurality of questions to the person and the processor is configured to analyze answers to the plurality of questions provided by the person to determine a memory strategy module for the person.

7. The system of claim 7, wherein the memory strategy module is configured to stimulate neural activity in the person.

8. The system of claim 1, wherein the first expected pattern comprises an anticipated pattern of use of the device and the first deviation comprises the person's use of the device in an unanticipated manner.

9. The system of claim 1, wherein the first expected pattern comprises a first rate of use of the device that is consistent with a predetermined rate and the first deviation comprises the person's use of the device at a second rate that is inconsistent with the first rate.

10. The system of claim 1, wherein the device is part of an internet of things system where the device is integrated within an appliance, furniture, and/or clothing of the person.

11. A method for managing cognitive decline of a person using a cognitive training device, the method comprising the steps of:
communicating, by a first processor, with a first information source comprising a device having a sensor configured to detect sensor data corresponding to a first behavior of the person, wherein the first behavior represents a first deviation from a first expected pattern;
communicating, by the first processor, with a second information source configured to receive, store, and transmit data corresponding to a second behavior of the person, wherein the data indicates the second behavior represents a second deviation from a second expected pattern;
receiving, by the first processor, the sensor data from the first information source and the data from the second information source and storing the sensor data and data in a database;
analyzing, by the first processor, the sensor data and the data to determine whether a predetermined threshold has been reached indicating the person is experiencing a first stage of cognitive decline;
generating, by the first processor, actionable information to assist a user in determining a treatment for the person to delay the progression of the first stage of cognitive decline;
determining, by the first processor, a memory strategy module as part of the treatment for the person based on answers to a plurality of questions provided by the person; and
implementing, by a memory strategy device, the memory strategy module to the person.

12. The method of claim 11, wherein the memory strategy module is configured to stimulate neural activity in the person.

13. The method of claim 11, wherein the first processor is further configured to request the sensor data be transmitted from the first information source to the database based on a frequency at which the person uses the device.

14. The method of claim 11, wherein the second information source comprises a user input device and a second processor configured to compare input from the person with input from the user and determine whether the inputs from the user and the person match.

15. The method of claim 11, wherein the first expected pattern comprises a completion of a task, an anticipated pattern of use of the device, or a first rate of use of the device that is consistent with a predetermined rate, and the first deviation comprises the person's failure to complete the task in its entirety, the person's use of the device in an unanticipated manner, or the person's use of the device at a second rate that is inconsistent with the first rate.
